(19) United States
(12) Patent Application Publication
(10) Pub. No.: US 2016/0061828 A1
(43) Pub. Date: Mar. 3, 2016

(54) GEL SENSOR

(71) Applicant: Seiko Epson Corporation, Tokyo (JP)

(72) Inventors: Kei HIRUMA, Chino (JP); Hiroshi YAGI, Tatsuno (JP)

(21) Appl. No.: 14/828,698

(22) Filed: Aug. 18, 2015

(30) Foreign Application Priority Data
Aug. 26, 2014 (JP) 2014-171843

(51) Int. Cl. G01N 33/543

(52) U.S. Cl. CPC G01N 33/54366

(57) ABSTRACT

A gel sensor includes a first region constituted by a material containing a stimulus-responsive gel and a second region containing fine particles at a higher content than the first region. It is preferred that the first region does not contain the fine particles. The gel sensor has the first region provided on the upstream side of the second region in the moving direction of a specimen, and when the area of the face of the first region on the upstream side is represented by S₁ (mm²) and the area of the face of the second region on the side facing the first region is represented by S₂ (mm²), it is preferred to satisfy the following relation: S₂≦S₁.

### BACKGROUND

1. Technical Field

The present invention relates to a gel sensor.

2. Related Art

A polymer gel (a stimulus-responsive gel) which contains fine particles, and expands or contracts in response to a given stimulus so as to change its color has been expected to be applied to a wide range of fields such as medical devices and optical device materials.

In such a stimulus-responsive gel, fine particles are required to be regularly arranged for obtaining Bragg reflection.

Therefore, in the case where a gel is formed merely using a mixture containing a polymer material (or a constituent unit thereof such as a monomer) constituting a stimulus-responsive gel and fine particles, the regularity of arrangement of the fine particles is low, and therefore, even when a given stimulus is received, a color change is not sufficient.

Meanwhile, JP-A-2010-139523 (PTL 1) discloses that a dispersion liquid in which particles are dispersed in an aqueous medium is concentrated by a reverse osmosis method, and the resulting concentrated liquid is used as a particle arrangement body in which the particles are regularly arranged (see claims and paragraph [0014] of PTL 1). Further, PTL 1 also discloses that by transforming the concentrated aqueous medium into a gel in the particle arrangement body, an immobilized particle arrangement body in which the particles are immobilized is formed (see paragraphs [0034] and [0035] of PTL 1).

By adopting the method described in PTL 1, the degree of color change when the stimulus-responsive gel receives a stimulus can be increased to some extent.

However, by the method described in PTL 1, the aqueous medium is contained in a given proportion in a concentrated state, so that the particle arrangement body has fluidity, and therefore, in this state, the arrangement state of the particles is changed and disturbed in some cases. Therefore, it is difficult to sufficiently increase the degree of color change when the stimulus-responsive gel receives a stimulus, and also a large degree of color change cannot be stably obtained.

### SUMMARY

An advantage of some aspects of the invention is to provide a gel sensor which changes its color stably and greatly by a given stimulus, and has excellent detection sensitivity and detection accuracy for the given stimulus.

Such an advantage is achieved by the invention described below.

A gel sensor according to an aspect of the invention includes a first region constituted by a material containing a stimulus-responsive gel and a second region containing fine particles at a higher content than the first region.

According to this configuration, a gel sensor which changes its color stably and greatly by a given stimulus, and has excellent detection sensitivity and detection accuracy for the given stimulus can be provided.

In the gel sensor according to the aspect of the invention, it is preferred that the first region and the second region are both in the form of a layer, and these regions are stacked on each other.

In the gel sensor according to the aspect of the invention, it is preferred that the gel sensor has the second region disposed closer to the side of an observer's viewpoint than the first region.

In the gel sensor according to the aspect of the invention, it is preferred that the gel sensor has a plurality of the first regions in the form of a layer and a plurality of the second regions in the form of a layer, and these regions are stacked on one another.

In the gel sensor according to the aspect of the invention, it is preferred that the gel sensor has a plurality of the second regions in which the contents of the fine particles are different from one another.

In the gel sensor according to the aspect of the invention, it is preferred that the second region has a part in which the content of the fine particles changes gradiently.

In the gel sensor according to the aspect of the invention, it is preferred that the second region has a part in which the content of the fine particles changes stepwise.

In the gel sensor according to the aspect of the invention, it is preferred that the gel sensor is in the form of a sheet, and the second regions are provided at different thickness positions in different in-plane parts of the gel sensor.

In the gel sensor according to the aspect of the invention, it is preferred that the first region does not contain the fine particles.

In the gel sensor according to the aspect of the invention, it is preferred that the gel sensor has the first region provided on the upstream side of the second region in the moving direction of a specimen, and when the area of the face of the first region on the upstream side is represented by S₁ (mm²) and the area of the face of the second region on the side facing the first region is represented by S₂ (mm²), the following relation is satisfied: S₂≦S₁.

In the gel sensor according to the aspect of the invention, it is preferred that the first region is provided on both faces of the second region in the form of a layer.

In the gel sensor according to the aspect of the invention, it is preferred that an absorbing member which absorbs a specimen is provided on a face on the opposite side to a face on the side of a supply source of a specimen in the gel sensor.

### BRIEF DESCRIPTION OF THE DRAWINGS

The invention will be described with reference to the accompanying drawings, wherein like numbers reference like elements.

FIG. 1 is a schematic longitudinal cross-sectional view for illustrating a gel sensor of a first embodiment.

FIG. 2 is a schematic longitudinal cross-sectional view for illustrating a gel sensor of a second embodiment.

FIG. 3 is a schematic longitudinal cross-sectional view for illustrating a gel sensor of a third embodiment.

FIG. 4 is a schematic longitudinal cross-sectional view for illustrating a gel sensor of a fourth embodiment.

### DESCRIPTION OF EXEMPLARY EMBODIMENTS

Hereinafter, preferred embodiments of the invention will be described in detail with reference to the accompanying drawings.

### Gel Sensor

Hereinafter, a gel sensor will be described.

### First Embodiment

First, a gel sensor of a first embodiment will be described.

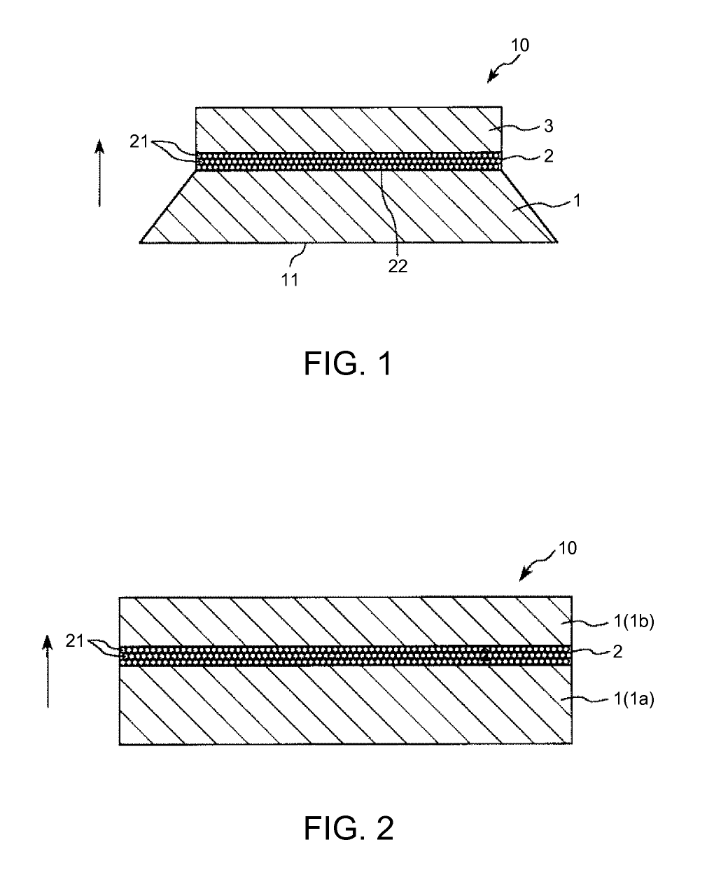

FIG. 1 is a schematic longitudinal cross-sectional view for illustrating a gel sensor of a first embodiment. In the following description, the upper side of FIG. 1 is referred to as “the side of an observer (the side of the viewpoint)” (the same shall also apply to FIGS. 2 to 4, which will be described later). Further, the arrow in the drawing indicates the moving direction of a specimen (the same shall also apply to FIGS. 2 to 4, which will be described later).

A gel sensor 10 includes a first region 1 which is constituted by a material containing a stimulus-responsive gel and a second region 2 which is provided in contact with the first region 1 and contains fine particles 21 at a higher content than the first region 1.

The stimulus-responsive gel constituting the first region 1 expands or contracts in response to a given stimulus.

The given stimulus to which the stimulus-responsive gel responds varies depending on the constituent material or the like of the stimulus-responsive gel, however, the examples thereof include various types of substances such as proteins, sugars, uric acid, lactic acid, various types of hormones, various types of ionic substances, and various types of metals; heat, and light.

The second region 2 is configured such that a plurality of fine particles 21 are arranged with high regularity, and contributes to the expression of a structural color by Bragg reflection.

Then, the second region 2 is configured to change a distance between the respective fine particles 21 accompanying the deformation (expansion or contraction) of the stimulus-responsive gel constituting the first region 1. As a result, it is configured such that the structural color caused by the second region 2 changes accompanying the deformation (expansion or contraction) of the stimulus-responsive gel constituting the first region 1.

As described above, by including the stimulus-responsive gel (a constituent component of the first region 1) which responds to a given stimulus and the fine particles 21 which contribute to the expression of a structural color by Bragg reflection in the gel sensor 10, the presence or absence of the stimulus, the intensity (amount or concentration) thereof, etc. can be detected.

In particular, the gel sensor 10 having the first region 1 and the second region 2 can prevent the arrangement of the fine particles 21 from being disturbed by undesirable flow of the fine particles 21, and thus can stably and greatly change its color by a given stimulus. As a result, excellent detection sensitivity and detection accuracy for a given stimulus can be achieved, and thus, the gel sensor 10 has excellent reliability.

The constituent materials of the first region 1 and the second region 2 will be described in detail later.

The forms of the first region 1 and the second region 2 are not particularly limited, however, in the configuration shown in the drawing, both are in the form of a layer, and these layers are stacked on each other.

According to this, while thinning the gel sensor 10, excellent adhesiveness between the first region 1 and the second region 2 is obtained, and the durability of the gel sensor 10 can be particularly enhanced. Further, while preventing the increase in the size of the gel sensor 10, the area capable of being visually recognized can be increased, and therefore, the detection of a given stimulus can be more easily performed.

Further, the effect of a change in the shape of the first region 1 can be more effectively reflected on the second region 2, and thus, particularly excellent detection sensitivity and detection accuracy for a given stimulus can be achieved.

The thickness of the first region 1 is preferably 20 μm or more and 5,000 μm or less, more preferably 50 μm or more and 4,000 μm or less.

According to this, while particularly enhancing the stability of the shape, durability, and reliability of the gel sensor 10, the deformation speed in response to a given stimulus can be increased.

The thickness of the second region 2 is preferably 0.03 μm or more and 30 μm or less, more preferably 0.1 μm or more and 15 μm or less.

According to this, while preventing the increase in the thickness of the gel sensor, the durability of the gel sensor 10 is particularly enhanced, and further, the discrimination of the structural color by Bragg reflection is more facilitated.

Further, in this embodiment, the gel sensor 10 has the second region 2 disposed closer to the side of an observer's viewpoint (on the upper side in the drawing) than the first region 1.

According to this, a change in the structural color in the second region 2 can be more favorable detected, and thus, particularly excellent detection sensitivity and detection accuracy for a given stimulus can be achieved.

Further, in this embodiment, the gel sensor 10 has the first region 1 provided on the upstream side of the second region 2 in the moving direction of a specimen (the arrow direction in the drawing), and when the area of the face (first face) 11 of the first region 1 on the upstream side of the specimen is represented by S₁ (mm²) and the area of the face (second face) 22 of the second region 2 on the side facing the first region 1 is represented by S₂ (mm²), it is preferred to satisfy the following relation: S₂≦S₁, it is more preferred to satisfy the following relation: 1.2≦S₁/S₂≦5.0, it is further more preferred to satisfy the following relation: 1.5≦S₁/S₂≦3.0.

By satisfying such a relation, while particularly enhancing the stability of the shape, durability, and the like of the gel sensor 10, a specimen incorporated from the first face 11 side can be efficiently supplied to the second region 2. As a result, particularly excellent detection sensitivity and detection accuracy for a given stimulus can be achieved, and the gel sensor 10 has particularly excellent reliability.

It is preferred that the relation of the areas as described above is satisfied in a state where the stimulus-responsive gel does not receive a given stimulus (for example, in the case where the stimulus-responsive gel is deformed in response to a specific component, in a state where the stimulus-responsive gel does not contain the specific component).

Further, in the gel sensor 10 of this embodiment, an absorbing member 3 which absorbs a specimen is provided on a face on the opposite side to a face on the side of a supply source of a specimen (on the lower side in the drawing).

According to this, for example, in the case where a specimen is sequentially supplied (in the case where a specimen is supplied continuously or intermittently), a previously supplied specimen is efficiently discharged from the region containing the stimulus-responsive gel (the first region 1), and a newly supplied specimen can be supplied to the region containing the stimulus-responsive gel (the first region 1), and therefore, a change in the stimulus amount over time can be found. That is, the detection of an accurate stimulus amount can be effectively prevented from being disturbed by mixing a previously supplied specimen and a newly supplied specimen. Further, in the case where a specimen is excessively supplied or the like, the gel sensor 10 can be prevented from getting excessively wet or the like due to the overflowing specimen.

Further, by including the absorbing member 3, for example, in the case where the supply of a liquid specimen is stopped or in the case where the supply amount of a specimen is significantly decreased, the stimulus-responsive gel can be effectively prevented from being dried when the humidity in the operating environment of the gel sensor 10 is low or the like. As a result, even in an environment where the stimulus-responsive gel is easily dried, the detection of a given stimulus can be stably performed with high reliability over a relatively long period of time.

In particular, in the configuration shown in the drawing, the absorbing member 3 is provided so as to cover the entire face of the second region 2.

According to this, the effect as described above can be more remarkably exhibited.

Examples of a preferred constituent component of the absorbing member 3 include a cellulose material and a water-absorbing polymer, however, a cellulose material is particularly preferred. Such a material has moderate hydrophilicity, and therefore, for example, in the case where a specimen contains water (for example, a body fluid such as sweat), the specimen can be favorably absorbed, and also water contained in the absorbed specimen can be favorably evaporated.

The thickness of the absorbing member 3 is preferably 0.1 mm or more and 2.0 mm or less, more preferably 0.2 mm or more and 1.5 mm or less.

According to this, while ensuring the visibility of the structural color, the effect of including the absorbing member 3 as described above is more remarkably exhibited.

Each of the respective regions constituting the gel sensor 10 may have a uniform configuration, or may have a plurality of parts having different configurations.

For example, the second region 2 may have a part in which the content of the fine particles 21 changes gradiently.

According to this, a color change occurs gradiently in the gel, and therefore, a change over time in the state of secretion of a stimulus substance can be ascertained.

Further, for example, the second region 2 may have a part in which the content of the fine particles 21 changes stepwise.

According to this, for example, in the case where an acceptable value or the like of a change in the stimulus amount has a plurality of thresholds, the generation levels of a given stimulus can be discriminated stepwise. More specifically, for example, in the case where a given stimulus is a component (a specific component) contained in a body fluid and the gel sensor 10 is a gel sensor which detects the specific component, the content of this specific component can be easily discriminated as to which of the following levels the content falls under: “a dangerous level”, “a cautious level”, and “a safe level”, and the physical condition management of a user of the gel sensor 10 can be more favorably performed.

When the gel sensor 10 is planarly seen, the area of a part where the second region 2 is provided is preferably 10 mm² or more and 360 mm² or less, more preferably 20 mm² or more and 180 mm² or less.

According to this, while more effectively preventing the increase in the size of the gel sensor 10, the detection of a given stimulus can be performed more easily with higher accuracy.

The thickness of the gel sensor 10 is preferably 0.2 mm or more and 7.0 mm or less, more preferably 0.3 mm or more and 5.5 mm or less.

According to this, while effectively preventing the increase in the size (thickness) of the gel sensor 10, the strength and durability of the gel sensor 10 can be particularly enhanced. Further, the detection of a given stimulus can be performed more easily with higher accuracy.

### Constituent Materials of First Region

Next, the constituent materials of the first region 1 will be described.

The first region 1 is constituted by a material containing a stimulus-responsive gel.

The stimulus-responsive gel may be constituted by any material as long as it responds to a given stimulus, but is generally constituted by a material containing a polymer material having a crosslinked structure and a solvent.

### Polymer Material

The stimulus-responsive gel contains a polymer material having a crosslinked structure.

The polymer material is an important component when the stimulus-responsive gel detects a specific component, and the structure thereof varies depending on the type of a component to be detected.

The polymer material constituting the stimulus-responsive gel is not particularly limited, and can be selected according to a component to be detected.

As the polymer material for detecting a specific component in the stimulus-responsive gel, various polymer materials are known, and for example, such known polymer materials can be also used.

Hereinafter, specific examples of the polymer material constituting the stimulus-responsive gel will be described.

As the polymer material constituting the stimulus-responsive gel, for example, a polymer material obtained by reacting a monomer, a polymerization initiator, a crosslinking agent, etc. can be used.

Examples of the monomer include acrylamide, N-methylacrylamide, N-isopropylacrylamide, N,N-dimethylacrylamide, N,N-dimethylaminopropylacrylamide, various quaternary salts of N,N-dimethylaminopropylacrylamide, acryloylmorpholine, various quaternary salts of N,N-dimethylaminoethylacrylate, acrylic acid, various alkyl acrylates, methacrylic acid, various alkyl methacrylates, 2-hydroxyethylmethacrylate, glycerol monomethacrylate, N-vinylpyrrolidone, acrylonitrile, styrene, polyethylene glycol diacrylate, 1,6-hexanediol diacrylate, neopentyl glycol diacrylate, tripropylene glycol diacrylate, polypropylene glycol diacrylate, 2,2-bis[4-(acryloxydiethoxy)phenyl]propane, 2,2-bis[4-(acryloxypolyethoxy)phenyl]propane, 2-hydroxy-1-acryloxy-3-methacryloxypropane, 2,2-bis[4-(acryloxypolypropoxy)phenyl]propane, ethylene glycol dimethacrylate, diethylene glycol dimethacrylate, triethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2-hydroxy-1,3-dimethacryloxypropane, 2,2-bis[4-(methacryloxyethoxy)phenyl]propane, 2,2-bis[4-(methacryloxyethoxydiethoxy)phenyl]propane, 2,2-bis[4-(methacryloxyethoxypolyethoxy)phenyl]propane, trimethylolpropane trimethacrylate, tetramethylolmethane trimethacrylate, trimethylolpropane triacrylate, tetramethylolmethane triacrylate, tetramethylolmethane tetraacrylate, dipentaerythritol hexaacrylate, N,N′-methylenebisacrylamide, N,N′-methylenebismethacrylamide, diethylene glycol diallyl ether, and divinylbenzene.

Further, examples of a functional group which can interact with a sugar include a boronic acid group (particularly, a phenylboronic acid group), and therefore, a monomer having a boronic acid group may be used. Examples of such a boronic acid group-containing monomer include acryloylaminobenzeneboronic acid, methacryloylaminobenzeneboronic acid, and 4-vinylbenzeneboronic acid.

In the case where an ionic substance (particularly, an ionic substance containing a calcium ion) is detected as a specific component, a crown ether group-containing monomer (particularly, a benzocrown ether group-containing monomer) such as 4-acrylamidobenzo-18-crown-6 ether, acryloyl aminobenzocrown ether, methacryloyl aminobenzocrown ether, and 4-vinylbenzocrown ether can be preferably used as the monomer.

In the case where an ionic substance such as sodium chloride is detected as a specific component, 3-acrylamidophenylboronic acid, vinylphenylboronic acid, acryloyloxyphenylboronic acid, N-isopropylacrylamide (NIPAAm), ethylenebisacrylamide, N-hydroxyethylacrylamide, or the like can be preferably used as the monomer. In particular, in the case where an ionic substance such as sodium chloride is detected as a specific component, it is preferred to use at least one monomer selected from the group consisting of 3-acrylamidophenylboronic acid, vinylphenylboronic acid, and acryloyloxyphenylboronic acid, and at least one monomer selected from the group consisting of N-isopropylacrylamide (NIPAAm), ethylenebisacrylamide, and N-hydroxyethylacrylamide in combination as the monomer.

In the case where lactic acid is detected as a specific component, 3-acrylamidophenylboronic acid, vinylphenylboronic acid, acryloyloxyphenylboronic acid, N-isopropylacrylamide (NIPAAm), ethylenebisacrylamide, N-hydroxyethylacrylamide, or the like can be preferably used as the monomer. In particular, in the case where lactic acid is detected as a specific component, it is preferred to use at least one monomer selected from the group consisting of 3-acrylamidophenylboronic acid, vinylphenylboronic acid, and acryloyloxyphenylboronic acid, and at least one monomer selected from the group consisting of N-isopropylacrylamide (NIPAAm), ethylenebisacrylamide, and N-hydroxyethylacrylamide in combination as the monomer.

The polymerization initiator can be appropriately selected according to, for example, the polymerization method thereof. Specific examples thereof include compounds which generate radicals by ultraviolet light including hydrogen peroxide, persulfates such as potassium persulfate, sodium persulfate, and ammonium persulfate, azo-based initiators such as 2,2′-azobis(2-amidinopropane)dihydrochloride, 2,2′-azobis(N,N′-dimethyleneisobutylamidine)dihydrochloride, 2,2′-azobis{2-methyl-N-[1,1-bis(hydroxymethyl)-2-hydroxyethyl]propionamide}, 2,2′-azobis[2-(2-imidazolin-2-yl)propane]dihydrochloride, 4,4′-azobis(4-cyanovaleric acid), 2,2′-azobisisobutyronitrile, 2,2′-azobis(2,4′-dimethylvaleronitrile), benzophenone, 2,2-dimethoxy-1,2-diphenylethan-1-one, 1-hydroxycyclohexyl phenyl ketone, 2-hydroxy-2-methyl-1-phenylpropan-1-one, 2,4,6-trimethylbenzoyl diphenylphosphine oxide, 1-[4-(2-hydroxyethoxy)-phenyl]-2-hydroxy-2-methyl-1-propan-1-one, and the like, and compounds which generate radicals by light with a wavelength of 360 nm or more such as substances obtained by mixing a thiopyrylium salt-based, merocyanine-based, quinoline-based, or styrylquinoline-based dye with 2,4-diethyl thioxanthone, isopropyl thioxanthone, 1-chloro-4-propoxythioxanthone, 2-(3-dimethylamino-2-hydroxypropoxy)-3,4-dimethyl-9H-thioxanthon-9-one mesochloride, 2-methyl-1[4-(methylthio)phenyl]-2-morpholinopropane-1,2-benzyl-2-dimethylamino-1-(4-morpholinophenyl)-butanone-1, bis(cyclopentadienyl)-bis(2,6-difluoro-3-(pyl-1-yl)titanium, or a peroxy ester such as 3,3′,4,4′-tetra-(t-butylperoxycarbonyl)benzophenone. Hydrogen peroxide or a persulfate can also be used as a redox-based initiator in combination with, for example, a reducing substance such as a sulfite or L-ascorbic acid, an amine salt, or the like.

As the crosslinking agent, a compound having two or more polymerizable functional groups can be used, and specific examples thereof include ethylene glycol, propylene glycol, trimethylolpropane, glycerin, polyoxyethylene glycol, polyoxypropylene glycol, polyglycerin, N,N′-methylenebisacrylamide, N,N-methylene-bis-N-vinylacetamide, N,N-butylene-bis-N-vinylacetamide, tolylene diisocyanate, hexamethylene diisocyanate, allylated starch, allylated cellulose, diallyl phthalate, tetraallyloxyethane, pentaerythritol triallyl ether, trimethylolpropane triallyl ether, diethylene glycol diallyl ether, and triallyl trimellitate.

The stimulus-responsive gel may contain a plurality of different types of polymer materials.

The content of the polymer material in the stimulus-responsive gel is preferably 0.7% by mass or more and 36.0% by mass or less, more preferably 2.4% by mass or more and 27.0% by mass or less.

### Solvent

By including the solvent in the stimulus-responsive gel, the above-mentioned polymer material can be favorably gelatinized.

As the solvent, any of various types of organic solvents and inorganic solvents can be used. Specific examples thereof include water; various types of alcohols such as methanol and ethanol; ketones such as acetone; ethers such as tetrahydrofuran and diethyl ether; amides such as dimethylformamide; chain aliphatic hydrocarbons such as n-pentane, n-hexane, n-heptane, and n-octane; alicyclic hydrocarbons such as cyclohexane and methylcyclohexane; and aromatic hydrocarbons such as benzene, toluene, and xylene, however, in particular, a solvent containing water is preferred.

The stimulus-responsive gel may contain a plurality of different types of components as the solvent.

The content of the solvent in the stimulus-responsive gel is preferably 30% by mass or more and 99% by mass or less, more preferably 50% by mass or more and 95% by mass or less.

### Another Component

The first region 1 may contain a component other than the above-mentioned components (another component).

Examples of such a component include an antioxidant, a UV absorber, an antifungal agent, an antibacterial agent, a deodorizer, and a refreshing component.

The first region 1 may contain the fine particles 21 as long as the content of the fine particles 21 is lower than in the second region 2, however, it is preferred that the first region 1 does not contain the fine particles 21.

According to this, the reliability of the gel sensor 10 as a whole can be prevented from being decreased by the expression of a structural color (a structural color with lower reliability than a structural color expressed in the second region 2) in the first region 1.

### Constituent Materials of Second Region

Next, the constituent materials of the second region 2 will be described.

### Fine Particles

The second region 2 is constituted by a material containing a plurality of fine particles 21.

The fine particles 21 contribute to the expression of a structural color by Bragg reflection.

Examples of a constituent material of the fine particles 21 include inorganic materials such as silica and titanium oxide; and organic materials (polymers) such as polystyrene, polyester, polyimide, polyolefin, poly(methyl(meth)acrylate), polyethylene, polypropylene, polyether sulfone, nylon, polyurethane, polyvinyl chloride, and polyvinylidene chloride, however, the fine particles are preferably silica fine particles. According to this, the fine particles have particularly excellent shape stability and the like, and thus, the durability, reliability, and the like of the stimulus-responsive gel can be particularly enhanced. Silica fine particles are relatively easily available as those having a sharp particle size distribution (monodispersed fine particles), and therefore are advantageous also from the viewpoint of stable production and supply of the stimulus-responsive gel.

The shape of the fine particles 21 is not particularly limited, but is preferably a spherical shape. According to this, the structural color caused by colloidal crystals is more favorably visually recognized, and the detection of a specific component can be more easily performed.

The average particle diameter of the fine particles 21 is not particularly limited, but is preferably 10 nm or more and 1,000 nm or less, more preferably 20 nm or more and 500 nm or less.

According to this, when a specific component is incorporated in the stimulus-responsive gel constituting the first region 1, that is, when an interparticle distance of the fine particles 21 constituting the second region 2 is changed accompanying the change in the shape (expansion or contraction) of the first region 1, the structural color caused by colloidal crystals is more easily visually recognized, and therefore, the detection and quantitative determination of a given stimulus can be more easily performed.

The “average particle diameter” as used herein refers to an average particle diameter on the volume basis, and can be obtained by, for example, measurement with a particle size distribution analyzer employing a Coulter counter method (model: TA-II, manufactured by Coulter Electronics, Inc.) using an aperture of 50 μm for a dispersion liquid obtained by adding a sample to methanol and dispersing the sample therein for 3 minutes with an ultrasonic disperser.

The second region 2 may include a plurality of different types of fine particles.

The content of the fine particles 21 in the second region 2 is preferably 50.0% by mass or more, more preferably 65.0% by mass or more and 98.0% by mass or less.

### Another Component

The second region 2 may contain a component other than the above-mentioned components (another component).

Examples of such a component include an antioxidant, a UV absorber, an antifungal agent, an antibacterial agent, a deodorizer, and a refreshing component.

Further, the second region 2 may contain a stimulus-responsive gel.

According to this, for example, the adhesiveness between the first region 1 and the second region 2 can be particularly enhanced. Further, when the deformation of the second region 2 occurs accompanying the deformation (expansion or contraction) of the first region 1, undesirable falling of some of the fine particles 21 constituting the second region 2 can be effectively prevented. As a result, the durability and reliability of the gel sensor 10 can be particularly enhanced.

In the case where the second region 2 contains a stimulus-responsive gel, the stimulus-responsive gel preferably satisfies the same conditions as those for the stimulus-responsive gel described as the constituent component of the first region 1.

In the case where the second region 2 contains a stimulus-responsive gel, the stimulus-responsive gel may have the same composition as that of the stimulus-responsive gel constituting the first region 1, or may have a different composition from that of the stimulus-responsive gel constituting the first region 1.

The content of the stimulus-responsive gel (the sum of the content of the polymer material having a crosslinked structure and the content of the solvent) in the second region 2 is preferably 0.1% by mass or more and 40% by mass or less, more preferably 1.5% by mass or more and 33.0% by mass or less.

According to this, aside from the first region 1, the effect of providing the second region 2 as described above is more remarkably exhibited, and also the durability of the gel sensor 10 can be particularly enhanced.

Further, by adopting a production method as mentioned below, the content of the stimulus-responsive gel in the second region 2 can be favorably controlled to fall within the above-mentioned range.

The gel sensor 10 as described above may be produced by any method, but can be produced by, for example, using a method including a first step of preparing a first material containing a stimulus-responsive gel and a film formed using a second material containing a plurality of fine particles 21, a second step of bringing the first material and the film into contact with each other and bonding these members to each other, and a third step (an absorbing member disposing step) of disposing an absorbing member 3 on a face on the opposite side to a face 22 facing the first material (first region 1) of the film (second region 2).

According to this, the gel sensor 10 which changes its color stably and greatly by a given stimulus, and has excellent detection sensitivity and detection accuracy for the given stimulus can be efficiently produced.

As the first material, for example, a stimulus-responsive gel material in the form of a sheet produced by synthesizing a polymer material using a component such as a monomer as described above can be used.

Further, as the first material, a material obtained by preparing a stimulus-responsive gel, followed by molding it into a given shape may also be used.

The film may be any as long as it contains a plurality of fine particles 21, and a film prepared by any method may be used, however, the film is preferably a film formed using a dispersion liquid containing the fine particles 21 and a dispersion medium for dispersing the fine particles 21.

According to this, the ease of handling (handleability) of the second material containing the fine particles 21 is enhanced, and thus, the productivity of the gel sensor 10 can be particularly enhanced. Further, the regularity of the arrangement of the fine particles 21 in the film to be formed can be easily enhanced, and thus, the detection sensitivity and detection accuracy for a given stimulus of the produced gel sensor 10 can be enhanced.

In this case, the film may contain the liquid component (dispersion medium) contained in the second material, however, it is preferred that the fine particles 21 have lost fluidity by at least partially removing the dispersion medium in the formation process.

According to this, the stability of the shape of the film can be enhanced, and thus, undesirable deformation and the like of the film (second region 2) in the subsequent second step or the like can be effectively prevented.

Example of a method for forming the film by removing the liquid component (dispersion medium) contained in the second material include a method in which the dispersion liquid supplied from a dispenser or the like is flattened with a flattening member such as a squeegee, followed by drying, and a method in which the dispersion liquid is ejected by an inkjet method or the like, followed by drying, however, an advection accumulation method is particularly preferred.

According to this, while enhancing the productivity of the gel sensor 10, the regularity of the arrangement of the fine particles 21 in the film to be formed can be particularly enhanced, and thus, the detection sensitivity and detection accuracy for a given stimulus of the gel sensor 10 to be produced can be made further excellent.

In addition, a film having a high density of the fine particles 21 can be formed, and therefore, the stability of the shape of the film is particularly enhanced. As a result, undesirable deformation and the like of the film (second region 2) in the subsequent second step or the like can be effectively prevented. Further, even if a pressing force in the second step is further increased, the occurrence of disturbance of the arrangement state of the fine particles 21 in the second region 2 can be effectively prevented, and therefore, while obtaining the above-mentioned effect, the adhesiveness between the first region 1 and the second region 2, and the like can be particularly enhanced, and the durability of the gel sensor 10 can be made particularly excellent.

The second material maybe any as long as it contains the fine particles 21, and may contain, for example, another component such as a liquid component (a dispersion medium) as described above, but preferably does not contain a polymer material constituting the stimulus-responsive gel. According to this, the regularity of the arrangement of the fine particles 21 in the film to be formed can be made particularly high.

In the second step, the first material and the film are brought into contact with each other and bonded to each other, whereby a stacked body having the first layer 1 and the second layer 2 is obtained.

In this step, apart of the gel material constituting the first material may be embedded in a space among the fine particles 21 constituting the film.

According to this, the adhesiveness (bonding strength) between the first region 1 and the second region 2 is particularly enhanced, and thus, the durability and reliability of the gel sensor 10 can be particularly enhanced.

This step can be favorably performed by applying a pressure, however, heating may be performed at the time of bonding. By doing this, for example, the viscosity of the first material is decreased, and a part of the gel material constituting the first material can be more favorably embedded in a space among the fine particles 21 constituting the film. Further, even if the pressing force is relatively decreased, the gel material can be embedded in a space among the fine particles 21, and therefore, the occurrence of disturbance of the regularity of the arrangement of the fine particles 21 in this step can be prevented.

The third step may be performed by merely placing the absorbing member 3 on the surface of the second region 2, or may be performed by adhering or fusing the absorbing member 3 to the second region 2, or the like.

### Second Embodiment

Next, a gel sensor of a second embodiment will be described.

FIG. 2 is a schematic longitudinal cross-sectional view for illustrating a gel sensor of a second embodiment. In the following description, different points from the above-mentioned embodiment will be mainly described, and the description of the same matter will be omitted.

As shown in FIG. 2, in a gel sensor 10 of this embodiment, first regions 1 (a first region 1a and a first region 1b) are provided on both faces of the second region 2, respectively, so as to sandwich the second region 2.

In this manner, a plurality of first regions 1 may be provided.

According to such a configuration, when the deformation of the second region 2 occurs accompanying the deformation (expansion or contraction) of the first region 1, undesirable falling of some of the fine particles 21 constituting the second region 2 can be effectively prevented. Further, an undesirable variation in the composition in the second region 2 (for example, a variation in the content of a stimulus-responsive gel, etc.) can be more effectively prevented, and thus, the occurrence of an undesirable variation in the deformation amount of the second region 2 accompanying the deformation (expansion or contraction) of the first region can be more effectively prevented. As a result, the durability and reliability of the gel sensor 10 can be particularly enhanced.

The first region 1a provided on the lower side of the second region 2 (on the specimen supply side) and the first region 1b provided on the upper side of the second region 2 (on the side of an observer's viewpoint) may satisfy the same conditions, or may satisfy different conditions.

### Third Embodiment

Next, a gel sensor of a third embodiment will be described.

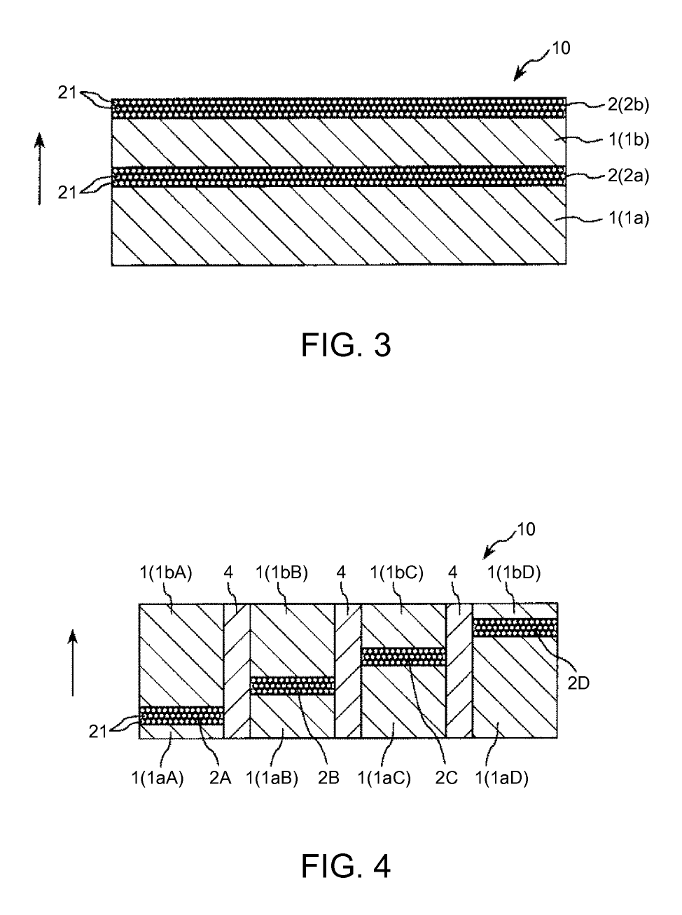

FIG. 3 is a schematic longitudinal cross-sectional view for illustrating a gel sensor of a third embodiment. In the following description, different points from the above-mentioned embodiments will be mainly described, and the description of the same matter will be omitted.

A gel sensor 10 of this embodiment has a plurality of first regions 1 in the form of a layer and a plurality of second regions 2 in the form of a layer, and these regions are alternately stacked on one another in the thickness direction. That is, a first region 1a, a second region 2a, a first region 1b, and a second region 2b are disposed in this order from the specimen supply side.

In this manner, not only a plurality of first regions 1, but also a plurality of second regions 2 may be provided.

According to such a configuration, the generation levels of a stimulus substance can be discriminated stepwise. That is, the color changes depending on to which region of the plurality of second regions 2 the change occurs, and therefore, the degree (intensity, amount, or the like) of a stimulus can be determined based on the color.

The plurality of second regions 2 of the gel sensor 10 may satisfy the same conditions, or may satisfy different conditions.

For example, the gel sensor 10 may have a plurality of second regions 2 in which the contents of the fine particles 21 are different from one another.

According to such a configuration, the same effect as described above is obtained.

### Fourth Embodiment

Next, a gel sensor of a fourth embodiment will be described.

FIG. 4 is a schematic longitudinal cross-sectional view for illustrating a gel sensor of a fourth embodiment. In the following description, different points from the above-mentioned embodiments will be mainly described, and the description of the same matter will be omitted.

A gel sensor 10 of this embodiment has a partition wall (a wall section) 4, with which a part in which the first region 1 and the second region 2 are provided such that these regions are overlapped with each other is divided into a plurality of cells each including the first region 1 and the second region 2. That is, the gel sensor 10 of this embodiment includes a cell (a first cell) having a first region 1aA, a second region 2A, and a first region 1bA overlapped in this order, a cell (a second cell) having a first region 1aB, a second region 2B, and a first region 1bB overlapped in this order, a cell (a third cell) having a first region 1aC, a second region 2C, and a first region 1bC overlapped in this order, and a cell (a fourth cell) having a first region 1aD, a second region 2D, and a first region 1bD overlapped in this order.

According to this, for example, by making the configurations of the respective cells different from one another, it becomes possible to detect a given stimulus under different conditions. For example, a plurality of different types of stimuli (given stimuli) can be detected.

Examples of a constituent material of the partition wall (wall section) 4 include polyolefins such as polyethylene, polypropylene, polybutadiene, and ethylene-vinyl acetate copolymers; polyesters such as polyvinyl chloride, polyurethane, polystyrene, polymethyl methacrylate, polycarbonate, polyamide, polyethylene terephthalate, and polybutylene terephthalate; acrylic resins such as polymethyl methacrylate; ABS resins, AS resins, ionomers, polyacetal, polyphenylene sulfide, polyether ether ketone, various types of rubber materials such as natural rubber, butyl rubber, isoprene rubber, butadiene rubber, styrene-butadiene rubber, silicone rubber, and fluororubber; silicone-based materials such as dimethylpolysiloxane; and various types of thermoplastic elastomers such as polyurethane-based, polyester-based, polyamide-based, olefin-based, and styrene-based thermoplastic elastomers.

Further, in this embodiment, in the gel sensor 10, by adjusting the thickness of the first region 1, the plurality of second regions 2 (2A, 2B, 2C, and 2D) are provided at different thickness (depth) positions of the gel sensor 10.

According to such a configuration, for example, times until the plurality of cells respond to a given stimulus contained in a specimen (times until the color changes) can be made different. As a result, for example, in the gel sensor 10 as a whole, a change over time with respect to the given stimulus can be favorably shown. More specifically, for example, in the case where the following relation: T_{A}≦T_{B}≦T_{C}≦T_{D} is satisfied, the amount (content) of a given stimulus contained in a specimen coming in contact with the first face 11 of the first region 1 at T_{A} seconds before an observation is made can be shown in the first cell (the second region 2A), the amount (content) of the given stimulus contained in a specimen coming in contact with the first face 11 of the first region 1 at T_{B} seconds before an observation is made can be shown in the second cell (the second region 2B), the amount (content) of the given stimulus contained in a specimen coming in contact with the first face 11 of the first region 1 at T_{C} seconds before an observation is made can be shown in the third cell (the second region 2C), and the amount (content) of the given stimulus contained in a specimen coming in contact with the first face 11 of the first region 1 at T_{D} seconds before an observation is made can be shown in the fourth cell (the second region 2D).

The width of each cell is preferably 1.0 mm or more and 20 mm or less, more preferably 2.0 mm or more and 10 mm or less.

According to this, while more effectively preventing the increase in the size of the gel sensor 10, a given stimulus can be detected more easily with higher accuracy.

Incidentally, the shapes, sizes (widths, etc.) of the respective cells may be the same or different.

### Application of Gel Sensor

The gel sensor can easily detect a given stimulus, and therefore, can be used as, for example a sensor for determining whether or not a specific substance is contained in a test subject (a specimen) or determining the concentration of a specific substance contained a test subject.

Further, the amount of a specific component incorporated in the stimulus-responsive gel can be easily detected, and therefore, the gel sensor can also be favorably used as a separation and extraction unit for separating and extracting a specific substance contained in a test subject. That is, at a stage where the amount of a specific component incorporated in the stimulus-responsive gel material is saturated or almost saturated, the contact thereof with the test subject is stopped, and according to need, it can be replaced by another gel sensor. According to this, the specific component can be collected from the test subject without waste.

More specific application of the gel sensor include, for example, sensors for biological substances (for example, various types of cells such as cancer cells and blood cells, proteins such as antibodies (including glycoproteins and the like), etc.), sensors for components (for example, lactic acid, uric acid, a sugar, etc.) contained in body fluids or substances secreted outside the body (for example, blood, saliva, sweat, urine, etc.), separation and extraction units for biological substances (particularly, trace biological substances and the like such as hormones), separation and extraction units for metals (particularly, rare metals, noble metals, etc.), sensors for antigens such as pollens (allergic substances), separation and extraction units for poisons, toxic substances, environmental pollutants, etc., sensors for viruses, bacteria, etc., sensors for components contained in soils, sensors for components contained in waste fluids (including drained water), sensors for components contained in foods, sensors for components contained in water (for example, salts and the like contained in brackish waters, rivers, paddies, etc.), cell culture monitors, and the like.

Further, the gel sensor is preferably a gel sensor to be used in close contact with the skin of a living body.

The skin of a living body generally has a complex rugged shape, however, the gel sensor has an excellent ability to follow a shape, and therefore can be favorably brought into close contact with the skin of a living body. Further, in the case where the gel sensor is used in close contact with the skin of a living body (for example, a case where a component contained in sweat is detected as a specific component when an exercise is performed, or the like), it is assumed that a large external force such as vibration or impact is applied to the gel sensor. However, even in such a case that a relatively large external force is applied to the gel sensor, a specific component (a given stimulus) can be accurately detected and can be easily discriminated. Therefore, the effect is more remarkably exhibited in the case where the gel sensor is used in close contact with the skin of a living body.

Further, the gel sensor can also favorably achieve size reduction and weight reduction. Accordingly, the gel sensor is suitable for use in the manner as described above.

Further, the gel sensor may be applied to a detection device equipped with a detection device which detects a change in the configuration (for example, volume, color, etc.) of the stimulus-responsive gel.

According to this, for example, even in the case where it is difficult to discriminate a change in the configuration of the stimulus-responsive gel with the naked eye (for example, a case where a color change or a volume change is small, a case where a reflected light with a variable wavelength is a light outside the visible light range, etc.), or in the case where the detection of a stimulus with higher accuracy (for example, quantitative detection, detection of a trace component, etc., requiring high accuracy) is demanded, the gel sensor can be favorably applied in such a case.

Hereinabove, preferred embodiments of the invention have been described, however, the invention is not limited thereto.

For example, in the above-mentioned embodiments, a case where the gel sensor is in the form of a sheet has been representatively described, however, the form of the gel sensor is not limited thereto, and may be any form, for example, a plate, a block, a string, a cylinder, a particle, or the like.

Further, the gel sensor may have a configuration other than the above-mentioned configurations. For example, the gel sensor may include an adhesive layer for attaching the gel sensor to a given position.

Further, the gel sensor may have one first region and one second region, and may not include other configurations.

Further, in the above-mentioned first embodiment, second embodiment, and fourth embodiment, the gel sensor has been described as a gel sensor in which the surface of the second region on the downstream side of a specimen (the side of an observer's viewpoint) is covered with an absorber or a stimulus-responsive gel, however, the surface may be covered with another member (for example, a member composed of any of various types or resin materials illustrated as a constituent material of the partition wall (for example, a film, etc.)). Also in such a case, the same effect as described above is obtained.

Further, in the above-mentioned third embodiment, a configuration in which two layers of the first region and two layers of the second region are provided and alternately stacked on each other has been described, however, in the case of having a stacked structure, the number of the first regions (the number of layers) and the number of the second regions (the number of layers) may be, for example, three or more.

Further, in the above-mentioned fourth embodiment, a case where a partition wall is provided between adjacent cells has been described, however, the partition wall may not be provided. For example, the gel sensor does not have a partition wall, and adjacent cells may be in contact with each other at their side surfaces, or a gap may be provided between adjacent cells.

Further, in the above-mentioned fourth embodiment, a case where the number of the cells is 4 has been representatively described, however, the number of the cells is not particularly limited, and may be, for example, 3 or less, or may be 5 or more.

The entire disclosure of Japanese Patent Application No. 2014-171843 filed Aug. 26, 2014 is expressly incorporated by reference herein.

What is claimed is:

1. A gel sensor comprising:
a first region containing a stimulus-responsive gel; and
a second region containing fine particles at a higher content than the first region.

2. The gel sensor according to claim 1, wherein the first region and the second region are both in the form of a layer, and these regions are stacked on each other.

3. The gel sensor according to claim 1, wherein the gel sensor has the second region disposed closer to the side of an observer's viewpoint than the first region.

4. The gel sensor according to claim 1, wherein the gel sensor has a plurality of the first regions in the form of a layer and a plurality of the second regions in the form of a layer, and these regions are stacked on one another.

5. The gel sensor according to claim 4, wherein the gel sensor has a plurality of the second regions in which the contents of the fine particles are different from one another.

6. The gel sensor according to claim 1, wherein the second region has a part in which the content of the fine particles changes gradiently.

7. The gel sensor according to claim 1, wherein the second region has a part in which the content of the fine particles changes stepwise.

8. The gel sensor according to claim 1, wherein
the gel sensor is in the form of a sheet, and
the second regions are provided at different thickness positions in different in-plane parts of the gel sensor.

9. The gel sensor according to claim 1, wherein the first region does not contain the fine particles.

10. The gel sensor according to claim 1, wherein the gel sensor has the first region provided on the upstream side of the second region in the moving direction of a specimen, and when the area of the face of the first region on the upstream side is represented by S₁ (mm²) and the area of the face of the second region on the side facing the first region is represented by S₂ (mm²), the following relation is satisfied: S₂≦S₁.

11. The gel sensor according to claim 1, wherein the first region is provided on both faces of the second region in the form of a layer.

12. The gel sensor according to claim 1, wherein an absorbing member which absorbs a specimen is provided on a face on the opposite side to a face on the side of a supply source of a specimen in the gel sensor.
